(19) United States
(12) Plant Patent Application Publication
(10) Pub. No.: US 2007/0220641 P1
(43) Pub. Date: Sep. 20, 2007

(54) Diascia plant named 'Diastured'

(75) Inventor: Henricus G.W. Stemkens, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/375,472

(22) Filed: Mar. 14, 2006

(51) Int. Cl. A01H 5/00

(52) U.S. Cl. PLT/263

(57) ABSTRACT

A new and distinct cultivar of Diascia plant named ‘Diastured,’ particularly distinguished by its red flowers and upright habit.

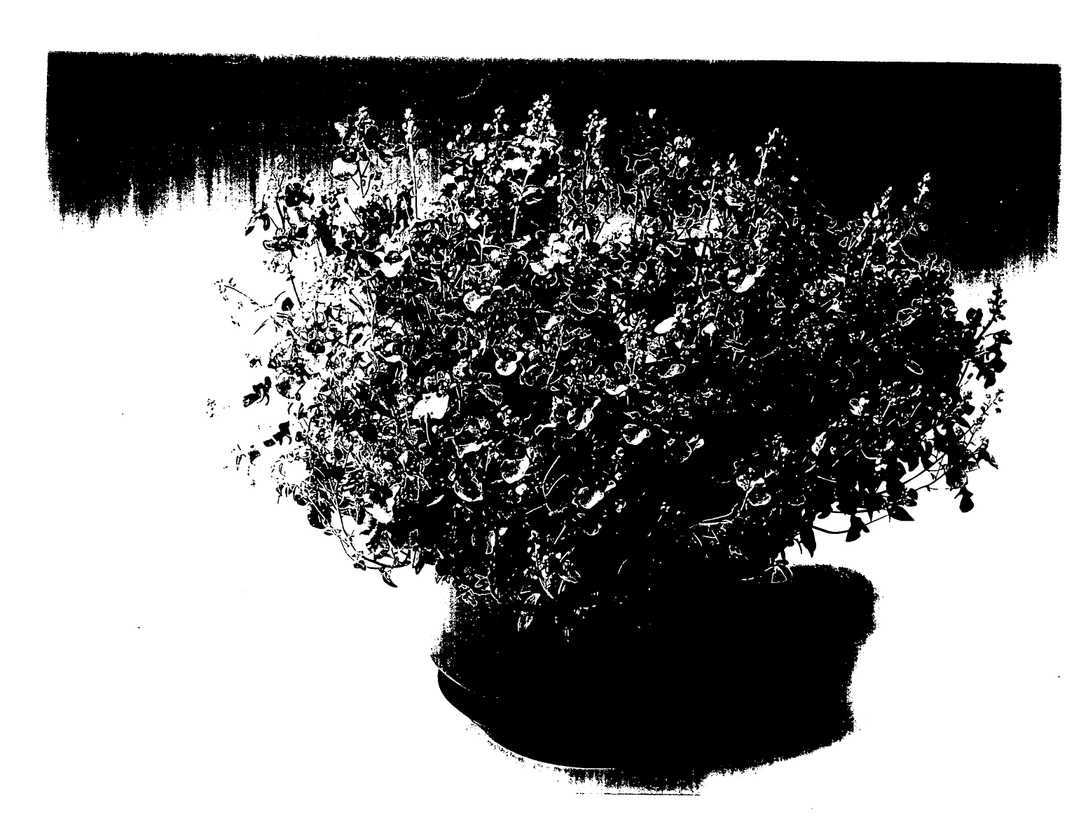

### LATIN NAME OF THE GENUS AND SPECIES OF THE PLANT CLAIMED

Diascia barberae

### VARIETAL DENOMINATION

‘Diastured’

### BACKGROUND OF THE NEW PLANT

The present invention comprises a new distinct cultivar of Diascia, botanically known as Diascia barberae.

The new cultivar is propagated from cuttings resulting from the cross of ‘Diaspetis’ and ‘C0008-1’. ‘Diaspetis’ is a rose flowering Diascia. ‘Diaspetis’ is commercially available as ‘Devotion Rose’ and is patented as U.S. Plant Pat. No. 15,579. ‘C0008-1’ is an apricot flowering Diascia. ‘C0008-1’ is not commercially available and is not known by any synonyms. ‘C0008-1’ has not been patented.

As a result of this cross the present cultivar was created in 2001 in Enkhuizen, Netherlands and has been repeatedly asexually reproduced by cuttings in Enkhuizen, Netherlands and Sarrians, France over a three-year period. It has been found to retain its distinctive characteristics through successive propagations. The new variety is stable and reproduces true to type in successive generations of asexual reproduction.

This new Diascia plant is an annual in most climatical zones in the US, only in zones 8, 9 and 10 it is a perennial plant.

### DESCRIPTION OF THE DRAWING

This new Diascia plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Diascia. The data which defines these characteristics were collected from asexual reproductions carried out in Enkhuizen, Netherlands. The plant history was taken on 10 week old plants, cultured in 10.5 cm containers, and blossomed under natural light in a greenhouse.

Color readings were taken in the greenhouse under ambient light. Color references are primarily to the RHS Colour Chart of The Royal Horticultural Society of London.

Differences between the new cultivar ‘Diastured,’ its
parents and a similar cultivar

| | ‘Diastured’ | ‘Diaspetis’ | ‘C0008-1’ | ‘Diastonia’ |
|---|---|---|---|---|
| Flower color | Red | Rose | Apricot | Deep rose |
| Plant shape | Upright | Upright | Trailing | Trailing |
| Flower size | Medium | Small | Large | Small |

‘Diastonia’ has a commercial name of ‘ Flying Colors Trailing Red’ and is patented as U.S. Plant Pat. No. 14,466.
The plant:
   Classification.—Botanical: Diascia barberae.
   Parentage.—Female parent: A seedling named ‘Diaspetis,’ U.S. Plant Pat. No. 15,579. Pollen parent: A seedling named ‘C0008-1’ is one of our seedlings from our C-generation of plants bred in 1997.
   Growth habit.—Upright and compact.
   Plantheight.—12-18 cm.
   Speading area of plant.—14-26 cm.
   Growth rate.—Vigorous.
   Strength.—Very good.
   Branching character.—Freely branching and lateral branching at every node.
   Quantity of branches.—60-100 branches per plant.
The stem:
   Diameter.—1-1.5 mm.
   Shape.—Round.
   Color.—137D.
   Length.—10-14 cm.
   Anthocyan pigmentation.—Absent.
   Length of internode.—Vegetative growth: 8-12 mm Generative growth: 10-20 mm.
   Pubescence.—Not pubescent.
The foliage:
   Phyllotaxis.—Opposite, decussate.
   Number of leaves per lateral stem.—20-40.
   Shape of blade.—Cordate.
   Texture.—Upper side: Smooth Lower side: Smooth.
   Attachment to leaf.—Petiolate.
   Venation.—Pinnate.
   Color of veins.—Upper side: 139C Lower side: 138D.
   Leaf margin.—Crenate.
   Leaf base.—Cordate.
   Leaf apex.—Acute to rounded.
   Length.—14-19 mm.
   Width.—12-14 mm.
   Depth of incision.—Less than 1 mm.
   Color.—Upper side: 139A Lower side: 138C.
   Pubescence.—No pubescence.
   Length of petiole.—2-3 mm.
   Diameter of petiole.—1-2 mm.
   Color of petiole.—139A.
   Petiole surface texture.—Smooth.
The bud:
   Peduncle length.—8-20 mm, depending on season.
   Peduncle diameter.—1-2 mm.
   Peduncle shape.—Long and threadlike.
   Peduncle color.—139A.
   Size of the bud.—Length: 3 mm Diameter: 3 mm.
   Shape of the bud.—Oval.
   Color of the bud.—184B.
   Number of buds per lateral branch.—12-35.
   Sepals.—Color both surfaces: 137A Number: 5, parted Length: 1-2 mm Width: 1-2 mm Shape: Elliptic, apex acute, base truncate Margin: Entire Texture: Smooth.
   Bracts.—Color upper side: 137A Color lower side: 137B Arrangement: Sessile Number: One underneath every pedicel Length: 1-2 mm Width: 1-2 mm Apex: Acute Base: Subcordate Margin: Entire Texture: Smooth.
   Pedicels.—Color: 183C Shape: Threadlike Length: 4-8 mm Diameter: Less than 1 mm.
The flower:
   Inflorescence length.—2-4 cm.
   Inflorescence diameter.—1-3 cm.
   Flower width.—16-20 mm.
   Flower length.—18-24 mm.
   Flower depth.—6-8 mm.
   Flower aspect.—Tipped upward and outward.
   Borne.—Solitary.
   Form.—Zygomorphic, five lobed, double nectar spur.
   Cluster.—Raceme.
   Color.—Upper surface: 58B. On the two upper banner petals there is a small yellow (color 9A) indentation just above the reproductive organs. Lower surface: 58C. On the two upper banner petals there is a small light yellow (color 9D) indentation at the base.
   Overlapping of the petals.—Separate.
   Number of petals.—Five: two upper banner petals, two lateral petals and one larger basal lip petal.
   Shape of the banner petals.—Apex rounded, base fused.
   Shape of the lateral petals.—Apex rounded, base fused, lower surface modified into nectar spurs.
   Shape of the basal lip petal.—Apex truncate, base fused.
   Petal margin of all petals.—Entire.
   Petal surface texture.—Smooth.
   Size of the banner petals.—Length: 3-4 mm Width: 4-6 mm.
   Size of the lateral petals.—Length: 2-3 mm Width: 3-5 mm.
   Size of the basal lip petal.—Length: 8-12 mm Width: 10-15 mm.
   Spur.—Length: 6-8 mm Diameter: 1-2 mm at petal attachment Color: 186A.
   Number of flowers per raceme.—25-35.
   Fragrance.—No fragrance.
   Lastingness of the bloom.—New florets continue to open in one raceme over a period of 14 days.
   Lastingness of one flower.—About 4 days depending on temperature.
   Flowering season.—Depending on climate from March until November.
The reproductive organs:
   Androecium.—Stamen number: Three Anther shape: Two-lobed Anther size: Less than 1 mm Anther color: Yellow 7B Amount of pollen: Very much pollen Pollen color: Yellow 7A.
   Gynoecium.—Pistil number: 1 Stigma shape: Rounded Pistil length: 2-3 mm Style length: 1-2 mm Style color: Green 143B Stigma color: Green white 157B Ovary color: Green 134A.
The fruit and the seed:
   Seed set.—No fruit development nor seed set has been observed.
The roots:
   Type of roots.—Fibrous.
   Rooting habit.—Fine and freely branching.
   Color.—Whitish, N155A.
Physiological and ecological characteristics:
   Disease/pest resistance.—Plants of the new Diascia have not been noted to be resistant to pathogens or pests common to Diascia.
   Temperature tolerance.—Plants of the new Diascia have been observed to tolerate temperatures from 0 to 35 degrees C.

What is claimed is:

1. A new and distinct cultivar of Diascia plant named ‘Diastured,’ as substantially illustrated and described herein.
